(19) United States
(12) Patent Application Publication
(10) Pub. No.: US 2014/0324186 A1
(43) Pub. Date: Oct. 30, 2014

(54) Medical Implants with Enhanced Osseointegration

(71) Applicant: B6 SIGMA, INC., Santa Fe, NM (US)

(72) Inventors: Gordon Daniel Blacklock, Albuquerque, NM (US); Vivek R. Dave, Concord, NH (US)

(21) Appl. No.: 14/358,243

(22) PCT Filed: Nov. 15, 2012
(86) PCT No.: PCT/US12/65228

§ 371 Date: May 15, 2014

Related U.S. Application Data
(60) Provisional application No. 61/559,991, filed on Nov. 15, 2011.

(51) Int. Cl. A61F 2/00; B23K 26/00; H01J 37/30; A61F 2/28

(52) U.S. Cl. CPC A61F 2/0077; A61F 2/28; B23K 26/0069; H01J 37/30
USPC 623/23.6; 148/714; 148/565; 219/121.35; 219/121.85; 250/492.3

(57) ABSTRACT

Medical implants with non-equilibrium surface structures are disclosed. The surface treatment of the implants greatly enhances osseointegration, reduces time to recovery following implant surgery, reduces surgery-related infections, and improves outcomes. The implants, including dental implants and other implants for insertion into or attachment to bone, are applicable to treatment of a wide variety of medical conditions. The methods of altering the surface properties of medical implants include exposure of a crystalline surface material, such as metal or ceramic, to a short burst of high thermal energy or shock, resulting in the introduction of a non-equilibrium concentration of crystal lattice defects in a surface layer.

### CROSS-REFERENCE TO RELATED APPLICATIONS

This application claims the priority of U.S. provisional application No. 61/559,991, filed on Nov. 15, 2011, which is incorporated herein by reference.

### BACKGROUND OF THE INVENTION

In dental and other hard bone implants, osseointegration is the direct structural and functional connection between ordered, living bone and the surface of a load-carrying implant (Branemark 1983). Titanium, which is used in many implants, cannot directly bond to living bone or other tissues. Therefore, the process of osseointegration may involve surface modification of titanium implants.

The majority of surface modification methods up to now have focused on increasing surface roughness or creating bioactive surfaces. Surface roughness provides a larger contact area between the metal implant and eventual bone cells. Bioactive surfaces have some effect on protein adsorption, but are more closely related to later adhesion of cells as well as bioactive elements, which could speed up the biological processes associated with wound healing.

The first generation of dental implants were machined (Branemark 1969) and exhibited reasonable osseointegration characteristics, but suffered from the tendency of the biomechanical stability to decrease over the first few weeks due to bone remodeling. During this process of bone remodeling, the bone necrosis is removed (Branemark 1997). This process is typically complete in 4 weeks, and then the stability of the implant increases steadily over the subsequent 16 weeks. Despite this 4 month healing time, machined implants have greater than 85% bone to implant contact, and successfully placed implants may last over a decade (Adell 1981, Branemark 1977).

The second generation of dental implants sought to modify the implant surface, and a wide variety of implant surface treatment strategies were developed. However, as observed by Ballo et al (2011), a fundamental understanding of the mechanisms of osseointegration and the specific ways in which surface treatments can accelerate osseointegration is incomplete. The second generation of implants used several surface modification strategies, including media blasting, acid etching, the combination of media blasting and acid etching, controlled oxidation or anodization, laser micro- and nano-texturing, and coatings of calcium phosphate, such as hydroxyapatite. Media blasting creates a randomized, rougher surface with both an increase in average surface roughness as measured by average peak height as well as a potentially greater peak to valley height of the surface features. Occasionally, particles of the blasting media may be embedded into the surface. Acid etching preferentially attacks grain boundaries, secondary phase particles, or any other site where there is a microstructural or surface energy inhomogeneity. There appears to be minimal effect from acid etching alone in the 0-2 week timeframe after implantation (Celletti et al 2006). However, acid etching after media blasting appears to remove residues and embedded particles from the blasting process, leaving behind a cleaner surface. By the mid 1990s the dental implant industry had standardized on media blasting followed by acid etching (Ballo et al 2011).

The third generation dental implants presently used has added further surface treatments in an effort to achieve shorter healing times and better osseointegration. One additional treatment has utilized storage of blasted and etched implants in dry nitrogen or sterilized saline solution to eliminate carbon contamination and improve hydrophillicity (Rupp et al 2006). Another such technique involves the creation of a biocompatible titanium hydride layer immediately on the surface of the titanium oxide (Conforto et al, 2004). Other techniques of “activating” blasted and etched implants include treatment with anions, fluoride treatments, or etching in hydrofluoric acid (Cooper et al. 2006). Through such combined mechanical and chemical processing, there have been observed improvements in osseointegration earlier in time, and significant improvements in the 6-12 week timeframe have been observed (Buser et al 2004 and Schwarz et al 2007). Some anodized implants are characterized by a partially crystalline layer enriched in various other ionic species and with an open surface pore structure in the 1-10 micron range. The structural and chemical properties can be altered by changing the anode potential, electrolyte composition, temperature, current, and type of ionic species transported in the solution (Lausma 2001, Hall and Lausma 2000, and Frojd et al 2008). In particular, phosphorous-containing anodized coatings have been shown to promote the early molecular events leading to osseointegration (Omar et al 2010). Laser micromachining has also been used to impart both micro-scale and nano-scale texture to an implant surface. The nano-structured surfaces appear to increase long-term interface strength through a coalescence between mineralized bone and the nano-textured surface features (Palmquist et al 2010) as well as increasing nearer-term removal torque (Branemark 2010). Various coatings have also been applied to implants. Plasma spray coatings of metal or calcium phosphate can improve interfacial strength (Cook et al 1987 and Carr et all 1995); however, they are also subject to poor long-term adherence of the coating as well as being prone to microbial infection (Rosenberg et al 1991 and Verheyen 1993). Sputter coatings are dense and uniform, but the process is slower than plasma spray and produces amorphous coatings which may then require subsequent heat treatment to recrystallize. Sputter coating may increase the short time fixation of the implant (3 weeks) but that at longer times (12 weeks) the difference between such coated implants and uncoated ones is negligible (Ong et al 2002). Biomimetic precipitation coatings seek to create calcium phosphate coatings using precipitation from a simulated biological fluid. In one study, in vivo osseointegration was compared for a variety of surface treatments, including uncoated titanium, plasma-sprayed hydroxyapatite, and biomimetically applied hydroxyapatite, all of which were statically indistinguishable (Vidigal et al 2009).

There remains a need to develop treatments for medical implants to promote more rapid and more reliable osseointegration.

### SUMMARY OF THE INVENTION

The invention provides medical implants with non-equilibrium surface structures which greatly enhance osseointegration, reduce time to recovery following implant surgery, reduce surgery-related infections, and improve surgical outcomes involving implantation of a medical device into bone or attachment to bone. The implants include dental implants and other implants for insertion into or attachment to bone. The invention further provides methods of treating a wide variety of medical conditions through implantation or attachment of a medical implant of the invention. The invention also provides methods of altering the surface properties of medical implants through exposure of a crystalline surface material, such as metal or ceramic, to a short burst of high thermal energy or shock, resulting in the introduction of a non-equilibrium concentration of crystal lattice defects in a surface layer.

One aspect of the invention is a method of treating a medical implant that has a crystalline surface material to promote protein adsorption to the surface material. The method includes the steps of heating the surface material to form a surface layer having crystal lattice defects, and thermally quenching the surface layer so as to preserve a non-equilibrium concentration of the defects.

Another aspect of the invention is a method of treating a medical implant having a crystalline surface material so as to promote protein adsorption to the surface material. The method includes the step of performing shock deformation of the surface to form a surface layer comprising a non-equilibrium concentration of crystal lattice defects.

Still another aspect of the invention is a medical implant containing a crystalline surface material. The surface material has been treated by a method that includes any of the above described methods.

Yet another aspect of the invention is a medical implant that includes a bulk material and a surface layer. The surface layer is disposed at a surface of the implant, and the bulk material is disposed beneath the surface layer. The bulk material and the surface layer have essentially the same chemical composition. The surface layer has a non-equilibrium concentration of crystal lattice defects, whereas the bulk material has an equilibrium concentration of crystal lattice defects.

Another aspect of the invention is a method of treating a medical condition. The method include the step of implanting into a patient in need thereof any of the medical implants described above.

For any of the above described implants and methods, in certain embodiments the implant is a dental implant.

### BRIEF DESCRIPTION OF THE DRAWINGS

FIG. 1 is a schematic representation of the arrangement of atoms at the surface of a crystalline material, showing vacancies (2. 3) and an interstitial atom (4).

FIG. 2 is a schematic representation of the preferential binding of adsorbate atoms to defect sites at a metal surface.

FIG. 3 is a schematic representation of the binding of a protein molecule to the surface of an implant material.

FIG. 4 is a schematic representation of the number of amino acid interactions in the binding of a protein molecule to the surface of an implant material.

FIG. 5 is a schematic representation of the interaction of a heat source (16) with the surface of a metal medical implant.

FIG. 6 is a schematic representation of the effect of a continuous heat source (19) moving across the surface of a metal medical implant.

FIG. 7 is an electron micrograph of the surface of a conventional dental implant. FIG. 8 is an electron micrograph of a dental implant that has been treated with a pulsed ion beam.

FIG. 9 is an electron micrograph showing a high magnification view of a dental implant that has been treated with a pulsed ion beam.

### DETAILED DESCRIPTION OF THE INVENTION

The critical phenomena occurring early in the osseointegration of a medical implant are heavily dependent on surface energy and adsorption of various chemical species to the surface of the implant that interfaces with bone. This invention provides novel methods to physically engineer and/or alter the surface energy, wettability, or adsorption characteristics of the implant material to enable better performance in terms of these physical phenomena and to ensure prompt and reliable osseointegration of the implant.

One aspect of the invention is a method of treating a medical implant so as to promote protein adsorption and osseointegration. A medical implant is treated at an exposed surface of a crystalline surface material of the implant. Treatment includes the application of a physical form of energy that is capable of causing structural defects and alterations in a surface layer of the crystalline surface material, and can also produce changes in the structure of the exposed surface itself. The changes in the surface layer and/or surface structure promote more rapid and more numerous adhesion of proteins and cells and accelerate the osseointegration process for implants that interface with bone.

The medical implant can be any physical structure intended for surgical implantation into the body of a human or animal patient. Especially preferred are medical implants that interface with bone when implanted in the patient, such as dental implants, joint replacements (e.g., hip, knee and other joint replacements inserted at one or more points into bone tissue), prostheses inserted into bone, and various types of surgical hardware such as screws, rods, or plates (e.g., for facial or skull reconstruction) that are designed for insertion into bone. The medical implant can be of homogeneous construction, i.e., composed of one type of material, either pure or an alloy or composite, or of heterogeneous construction, i.e., composed of different parts or sections having different types of materials. The medical implant and/or the crystalline surface material, can include or essentially consist of a metal or ceramic material, such as titanium, titanium oxide, alloys including titanium; zirconium, zirconium oxide, alloys including zirconium; aluminum, aluminum oxide, alloys containing aluminum; cobalt-chromium alloys; any of the 300 series stainless steels; any of the 400 series stainless steels; calcium phosphate, or hydroxyapatite; or any combination thereof. The crystalline surface material can be a separate material that is bonded with a substructure or bulk material of the implant, or the crystalline surface material can be a surface region of a homogeneous or bulk material of the implant. The crystalline surface material contains a substantial fraction of a crystalline phase. Preferably, the crystalline surface material is substantially entirely crystalline at least in a region at and near a surface of the implant to be treated to promote osseointegration. However, the crystalline surface material can contain an equilibrium concentration of crystal lattice defects prior to treatment according to the invention. After treatment according to a method of the invention, the crystalline surface material will contain at least a portion that has a non-equilibrium concentration of crystal lattice defects. Crystal lattice defects for use in the invention include vacancies, dislocations, disinclinations, steps, and grain boundaries. Vacancies are a preferred crystal lattice defect.

The treatment according to the invention may be applied over the entire surface of the implant or only over one or more portions of the surface. Preferably, the treatment is applied over the full extent of the implant surface that is intended to interface with bone. The treated surface includes or is entirely composed of the crystalline surface material as described above. Treating the surface includes applying an energy source to the crystalline surface material so as to rapidly heat or shock the material.

Suitable energy sources that heat the material include pulsed or continuous ion beams, pulsed or continuous electron beams, pulsed or continuous lasers, and electric arcs. Energy sources that induce a shock wave in the material include pulsed lasers, pulsed ion beams, and pulsed electron beams. Shock energy sources can be differentiated from heat sources in that the creation of defects by shock is caused by homogeneous nucleation of the defects directly through the application of a high applied stress (i.e., a shock wave). This is a physically different mechanism from thermally generated crystalline defects, which follow the Arrhenius rate equation for thermally activated phenomena. Shock waves produce mechanically induced defects without appreciable heating. The shock produces a pressure pulse which directly increases the internal energy of the solid through a material constant known as the Grueneisen constant. This rise in internal energy over very short time periods allows the spontaneous nucleation and formation of lattice defects. Furthermore, shock deformation produces a very dense dislocation tangle network in its wake—an additional source of crystal defects in the shocked region that resists annealing during subsequent heat treatment and is far in excess of the defect density possible using conventional means of surface deformation such as rolling, burnishing, or media blasting (Leslie 1973).

The energy input to the crystalline surface material is generally of high intensity and brief duration. For example, an ion beam or electron beam source is preferably operated in the range of 100 kV (kilovolts) to 10 MV (megavolts) for the beam voltage, 1 A (amp) to 1000 A for the beam current, and is applied to the crystalline surface material for a duration of about 10 nanoseconds to about 100 microseconds. A pulsed laser pulsed can have a pulse width from about 10 picoseconds to about 1 millisecond, a pulse energy from about 1 picojoule per pulse to about one joule per pulse, wavelength from about 375 nm to about 1550 nm, and a pulse repetition rate from a single pulse to about 1 million pulses per second. Multiple pulses can be delivered either as one continuous train of pulses, or as plural packets separated by a rest interval. In order to produce a shock wave by pulsed laser, the pulses must be spaced such that there is sufficient time for the material to cool between pulses, or to avoid excessive heating so that the pulsed mechanism of generating defects using shock waves does not default to the thermal method of generating defects using rapid heating and quenching. This time delay may be estimated from the characteristic heat conduction time, which is given by:

t=(x²/4α) (1)

where x is a characteristic length in cm and α is the thermal diffusivity of the material in units of cm²/s. For example, for titanium, the thermal diffusivity is 0.042 cm²/s. The characteristic length depends on both the surface area irradiated by the beam as well as the average depth of the zone directly affected by the heat source in question. Therefore, the cube root of this affected volume may be taken as a representative characteristic length. For example, using a pulsed ion or electron beam with an energy of 500 keV, the spot size may be as large as 5 mm, but the depth directly affected by the laser will be very shallow and will be on the order of 1 to 5 micrometers or less (Dave, 1995). Therefore the characteristic volume for this case is the area of the spot times the depth, or 1×10⁻⁴ cm³. Taking the cube root, we get 0.05 cm as a characteristic length. Therefore using Equation 1 we calculate that to avoid excessive heating, it would be preferable to space pulses such that the pulse interval is no shorter than 0.015 seconds, which corresponds to a maximum pulse frequency of 67 Hz. If the heat sink conditions are not ideal and heat extraction from the implant is poor, it is preferable to pick a much lower pulse rate than the highest allowable to ensure proper removal of heat in between pulses and to make sure the process operates in a purely mechanical as opposed to thermal regime for defect generation.

Treatment with a high energy beam or laser is preferably carried out in an environment that is essentially oxygen free, such as in a vacuum or in the presence of an inert gas (e.g., nitrogen, argon, or another inert or noble gas, or a mixture of such gases). The presence of oxygen during the treatment would produce undesirable chemical reactions, such as the formation of oxides, which could alter the implant material or its crystalline structure.

The surface layer to be treated, or formed during treatment, is a thin layer located at an exposed surface of the implant. The surface layer can have a thickness from about 10 nanometers to about 25 microns, or is less than about 10 microns. A surface layer thickness of about 10 microns is preferred. A surface layer that is too thin will not provide a significant effect on protein adsorption or osseointegration, whereas a surface layer that is too thick can alter the physical properties of the implant, or could promote delamination from the bulk material of the implant.

In one embodiment of a method of treating a medical implant, the implant is first treated with an energy source as described above so as to heat the crystalline surface material and form a surface layer that includes crystal lattice defects. The surface layer material can include an equilibrium concentration of crystal lattice defects prior to heating, where the equilibrium concentration is established by the process of fabricating the implant (e.g., by an ordinary metal annealing process). However, during the heating step, a higher, non-equilibrium concentration of crystal lattice defects is produced in the surface layer. Following the heating step, the surface layer is thermally quenched so as to preserve a non-equilibrium concentration of crystal lattice defects in the surface layer. The heating step is accomplished using one or more of the energy sources described above. The quenching step is necessary in order to prevent the material from re-establishing equilibrium and thus to preserve the non-equilibrium concentration of defects. Thus, thermal quenching must be more rapid than the approach to equilibrium. The speed of thermal quenching can be calculated as described below. In many cases, sufficiently rapid thermal quenching can be accomplished by rapidly removing or turning off the energy source and allowing the thermal energy within the surface layer to dissipate within the attached bulk material by conduction.

Equilibrium and non-equilibrium concentrations of such defects can be calculated based on the material and conditions used, as described below. These concentrations also can be directly measured by visualization of the crystalline structure of the treated or untreated surface layer, or a portion thereof, using transmission electron microscopy. For example, a thin section of the surface layer can be cut out using an ion beam, and the section can be placed into a transmission electron microscope, with which the individual atoms and their arrangement can be seen. Using the electron microscope, defects in the lattice structure can be counted in a known volume of the material to give the defect concentration.

In another embodiment of treating a medical implant, shock deformation of a crystalline surface material is performed. The shock deformation creates a shock wave in the crystalline surface material, resulting in the mechanically-induced formation of a non-equilibrium concentration of crystal lattice defects in a surface layer of the material.

The foregoing methods of treating a medical implant may induce surface structural alterations in addition to producing a non-equilibrium concentration of crystal lattice defects. Typically, the surface structural modifications result in the smoothing of the surface, with the elimination of all or nearly all surface features (i.e., structures protruding above the surface plane) and surface voids (i.e., structures dipping below the surface plane) having an average or root mean square size (i.e., diameter) of about 10 microns or less, or in some embodiments 5 microns or less. Surface features or voids larger than about 5 or 10 microns in size remain generally unaffected, and are available as a residual surface roughness that can be useful in promoting osseointegration. However, removal of small surface features and voids can improve the performance of the implant by removing points where contamination with inorganic or organic matter (including microbes) can accumulate. The smoothing of a medical implant surface on a fine length scale (i.e., removal of surface features of less than 5 or 10 microns in size) is thought to contribute significantly to reducing inflammation following implantation surgery. Such small surface features are potential sites for trapping both organic and inorganic contamination. Their removal thus reduces the extent of the immune response required to eliminate these contaminants. The treated implant therefore presents a cleaner or more readily cleanable surface. The surface structure of a medical implant can be determined using scanning electron microscopy (SEM) or atomic force microscopy. Comparison of the surface structure using these methods before and after the treatment process can be used to determine the effects of the treatment on surface structure.

A characteristic of the methods of treating a medical implant according to the invention is that they result in the formation and preservation of a non-equilibrium concentration of crystal lattice defects in a surface layer. In some embodiments of these methods, the surface layer formed by the treatment is disposed at the surface of the implant, and resides above and optionally in direct contact with a bulk material layer that does not have a non-equilibrium concentration of crystal lattice defects, but instead has an equilibrium concentration of crystal lattice defects, or is essentially devoid of crystal lattice defects.

The present invention also contemplates medical implants made by any of the foregoing methods of treating the surface of the implant. Medical implants according to the present invention have several advantageous properties. They possess a surface layer which contains a non-equilibrium concentration of crystal lattice defects. Those defects, which are present at a higher concentration than found in conventional implants, promote more rapid and more extensive protein binding to the implant, which in turn leads to cell attachment and eventually to the firm attachment of bone tissue to the implant (osseointegration) as well as adhesion to neighboring tissues such as skin, cartilage, connective tissue, and other tissues. The surfaces of conventional implants contain only an equilibrium concentration of crystal lattice defects, because they are made by processes that allow the concentration of such defects to reach equilibrium. The equilibrium concentration of defects in near surface layers of conventional implants promotes protein adhesion, cell adhesion, osseointegration, and tissue adhesion to a lesser degree than the implants of the present invention.

Another aspect of the invention is a method of treating a medical condition. The method includes implanting into a human or animal patient, such as by surgery, a medical implant that has been treated by any of the methods of the invention. The method can include further steps, such as testing for or monitoring the progress of osseointegration or the presence of infection after the implant surgery. The medical condition can be any condition requiring the use of a medical implant in a patient, such as an implant that interfaces with bone. Thus, the medical condition can be the insertion of a dental implant into a jaw bone, the replacement of a joint such as a hip joint, or the repair of a bone fracture.

The methods described herein encompass a wide range of length scales and physical phenomena ranging from the sub-angstrom level to the millimeter scale. Examples include: the fundamental physics of metals (angstroms); surface thermodynamic and quantum-mechanical properties (angstroms and below); molecular processes at bone contact surfaces (angstroms to nanometers); protein adsorption to bone contact surfaces (nanometers); cellular attachment to protein scaffolds (micrometers); and bone growth, mineralization, and eventual attainment of full biomechanical stability of the implant (micrometers to millimeters).

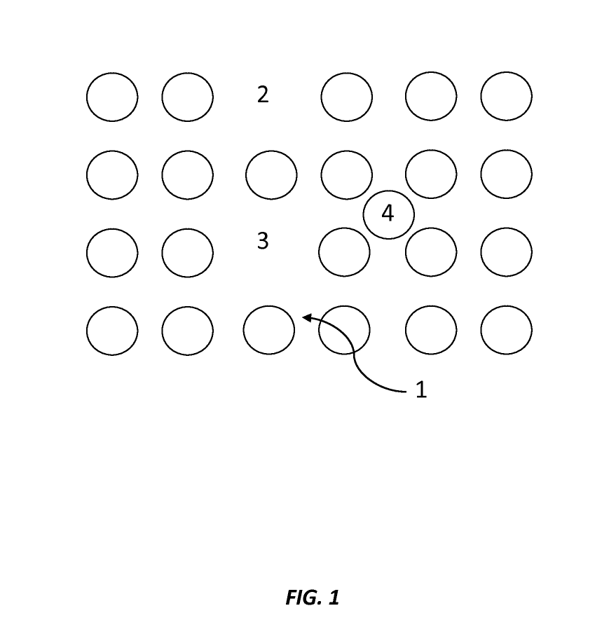

The bulk energy and the surface energy of a crystalline surface material can be altered by the presence of crystalline defects. One such defect is called a vacancy. Crystalline vacancies are the absence of an atom on a lattice. This is shown in FIG. 1 in the case of a two dimensional lattice as an example. The crystal lattice 1 is a regular array of atoms with a specified symmetry. Vacancies can either occur at the surface 2 or in the interior 3 of the crystal. Location 4 in FIG. 1 represents an interstitial atom, or an atom that has been displaced from the lattice and now occupies a site between other atoms, or an interstitial site. When a vacancy forms, the atom that used to occupy the vacancy site either becomes an interstitial atom or jumps to another vacancy site. This combination of a vacancy and an interstitial is known as a Frenkel Pair (Kittel 2005).

Vacancies have a specific energy of formation. At a given temperature, there is an equilibrium concentration of vacancies as specified by conditions of thermodynamic equilibrium. This concentration is given by (Haasen 1986):

$${C}_{V} = exp [ \frac{{S}_{VF}}{k} ] exp [ \frac{- {E}_{VF}}{kT} ] \qquad ( 2 )$$

where C_{V} is the equilibrium concentration of vacancies at absolute temperature T, E_{VF} is the free energy of formation of a vacancy, S_{VF} is the entropy of formation of the vacancy, and k is the Boltzmann constant.

When a metal surface is suddenly subjected to a large temperature pulse over a very short period of time, the equilibrium vacancy concentration would rise quickly according to equation 1. This rapid heating will create Frenkel Pairs. In order to determine how fast these Frenkel pairs can form on rapid heating of a crystal, it is first necessary to estimate the time needed to get to equilibrium. The first assumption for an equilibrium time might be made based on the phonon spectrum of the crystal. The phonon spectrum, or the total assembly of quantized states of vibration of a crystal, approaches equilibrium on the order of 10⁻¹¹ sec (Seitz and Koehler 1956). Point defects however take longer to form since there has to be a vibration fluctuation of sufficient strength to knock the atom out of its lattice position. An equation describing the rate of Frenkel pair generation is given by (Koehler and Lund 1965):

$$\frac{d {C}_{PAIR}}{d t} = 6 ω \cdot exp ⌈ \frac{- {E}_{PAIR}}{kT} ⌉ \qquad ( 3 )$$

where C_{PAIR} is the concentration of Frenkel pairs, w is a characteristic atomic vibration frequency which is typically taken to be 10¹³ (Kittel 2005), E_{PAIR} is the energy of pair formation, T is the absolute temperature, and k is the Boltzmann constant. For titanium, the energy of formation of a vacancy in Equation 1 is 1.2 electron volts or eV (Novikov et al 1980), but the energy of formation of a Frenkel pair in Equation 2 is higher, between 3 and 5 eV (He and Sinnott 2005). Assuming that the temperature to which the metal surface is raised is 1273 K (1000 C), then according to equation 2 the rate of generation of Frenkel pairs is approximately 79.6 per second. The equilibrium concentration of Frenkel pairs is given by (Koehler and Lund 1965):

$${C}_{PAIR} = exp [ \frac{- {E}_{PAIR}}{2 kT} ] \qquad ( 4 )$$

Substituting in the values shown above, the equilibrium concentration is approximately 1×10⁻⁶ or 1 ppm. Therefore, dividing this number by the Frenkel pair generation rate, the equilibrium time is estimated to be 1.45×10⁻⁸ seconds, or on the order of tens of nanoseconds. At lower temperatures, this equilibrium time will be longer. For example, at 700 C or 973K, this equilibrium time is on the order of 1 microsecond. At the melting point of titanium, the equilibrium time is much shorter, and is approximately 1×10⁻¹⁰ sec, or a tenth of a nanosecond. Therefore it is seen that the time required to reach an equilibrium concentration of vacancies varies from a tenth of a nanosecond at the melting point to close to a microsecond at one half the melting point.

After a supersaturation of vacancies is created, the vacancies tend to anneal out to try to reduce the free energy of the system. Alternatively they tend to form lower energy cluster configurations. Therefore, the faster the quench rate, the more likely the supersaturated vacancy concentration is to be preserved. Kauffman and Smith (1965) have studied this effect and found that a quenching temperature or cooling rate of 10⁴ degrees K per second or higher would be expected to avoid excessive vacancy annealing and to maintain the highly non-equilibrium microstructure produced in the method of the present invention.

Surface defects such as steps, vacancies, dislocations, disinclinations, and grain boundaries are known to play an important role in controlling the mechanical and chemical properties of surfaces. With regards to adsorption, modification of the electronic density and the electronic potential around defects leads to different adsorption behavior as compared to the rest of the surface (Persson 1992).

The adsorption of atoms and molecules to defect sites can be understood qualitatively by considering that adsorbed atoms help to “complete” the missing electron density at the defect sites. This can be done either through an electrostatic interaction, such as physical adsorption through weak van der Waals forces, or chemisorption through the formation of covalent bonds between adsorbed atoms and a metal (or metal oxide) surface (Feibelman et al 1996). Some previous work (Vakarin 2003) has found a correlation between the number density of free defects and the adsorbate cluster size. Such a specific size effect modifies the thermodynamic and kinetic properties; for example, the adsorbate surface diffusion coefficient is proportional to the density of free defects.

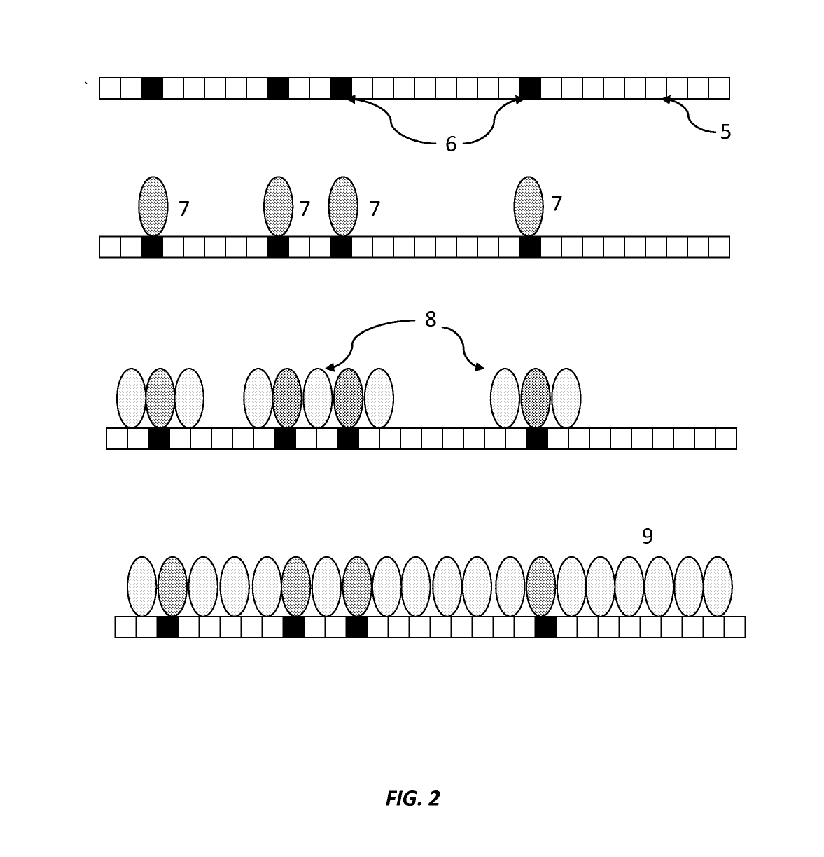

The preferential binding of adsorbate atoms to defect sites is schematically illustrated in FIG. 2. In the first view, the surface of the metal is schematically portrayed as a lattice consisting of sites with no defects 5 and defect sites 6. Adsorbed molecules 7 first attach to the defect sites. As more molecules adsorb to the surface, the adsorbed layer grows out in islands 8 from the initial nucleation sites and begins to fill up the surface. Eventually the entire metal surface is covered with adsorbed molecules 9.

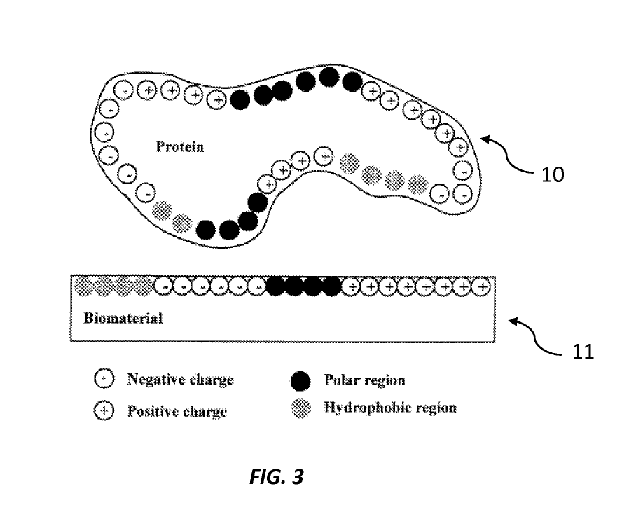

Protein adsorption is dominated by considerations of surface energy, intermolecular forces, ionic, and electrostatic interactions, in addition to considerations of hydrophobictiy or hydrophillicity. Proteins are biological heteropolymers that are composed of amino acid units. Certain amino acids have a side chain that may be charged based on the details of the solution chemistry (e.g., pH, ionic strength) and environment it is in. It is through these charged amino acid side chains that proteins are able to adsorb to multiple sites on a metal surface (Dee 2002). As shown schematically in FIG. 3, protein adsorption is dependent on both the properties of the molecule 10 as well as of the surface 11. See Dee (2002).

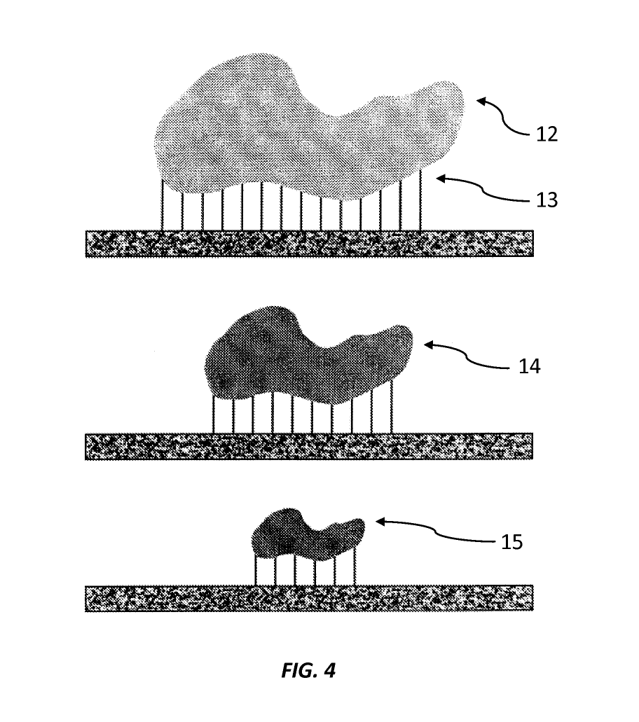

With respect to protein adsorption on medical implant surfaces, the effect of surface roughness is well-documented and leads to approximately a 30%-50% increase in the adsorption kinetics (Rechendorff 2006). The interaction of a protein molecule with a surface is dominated by the amino acid interactions. A larger protein 12 has more links with the surface made by the amino acids 13 as compared to smaller proteins 14 and 15. This is schematically shown in FIG. 4. It is expected that the increased surface energy will provide more energetically favorable sites or activated sites for amino acids to attach to, again speeding up the kinetics of protein adsorption. Alternatively, in proteins that are folded but have less stability of the folded state, the increase in the number of surface active sites may in fact encourage folded proteins to unfold and become adsorbed to the surface through additional amino acid chain interactions. See Dee (2002).

Wound healing around a dental implant is characterized by three distinct phases: initial formation of blood clot occurs through a biochemical activation, followed by cellular activation, which in turn is followed by a cellular response (Stanford and Schneider 2004). The various events occurring at the implant interface include the following (see also Anil et al 2011 and Ramazanoglu et al 2011):
1) Dental implant surfaces come into contact with various blood components during surgery.
2) The blood wets the dental implant much as any viscous fluid will wet a metal surface.
3) Various proteins get adsorbed onto the metal surface.
4) The most important of these proteins is human plasma fibrinogen (HPF), which partakes in blood coagulation, facilitates adhesion and aggregation of platelets (Silvennoinen et al, 2011).
5) Fibrinogen is converted to fibrin.
6) Complement and kinin systems get activated.
7) Migration of bone cells will occur through the fibrin of a clot.
8) Since fibrin is adherent to the metal surface, migration of osteogenic cell populations towards the metal surface will occur.
9) Migration of these cells will however cause retraction of the fibrin scaffold.
10) Therefore the ability of the fibrin scaffold to strongly adhere to the metal surface during the phase of wound contraction is critical to determining whether or not the migrating cells will reach the implant surface.
11) Platelets are activated as a result of interactions of platelets with the implant surface.
12) Thrombus formation and blood clotting results.
13) Platelets are the source for growth and differentiation factors which act as signaling molecules for recruitment and differentiation of undifferentiated mesechymal stem cells at the implant surface.
14) Other proteins which could promote cell adhesion and osseointegration include fibronectin and vitronectin.
   This list is not intended to provide a complete picture of all of the possible processes involved during the osseointegration process.

The time dependence of these various physical, chemical and biological processes for a dental implant is shown in Anil et al 2011. Despite the considerable complexity of this physical-chemical-biological sequence of events, the essential steps within the first six days after implantation are wetting of the metal surface by blood and plasma components and the concomitant adsorption of proteins to the metal surface. Both of these phenomena are heavily influenced by surface energy and surface science considerations.

In view of this osseointegration process, three objectives of the present invention are: 1) to improve wetting and wettability of the surface through alteration of the surface energy by introduction of a non-equilibrium concentration of crystal lattice defects; 2) to improve adsorption of proteins through creation of a non-equilibrium surface concentration of defects which serve as preferred adsorption sites; and 3) to smooth the surface at dimensions less than several microns so as to seal up sites which could trap organic and inorganic contaminants.

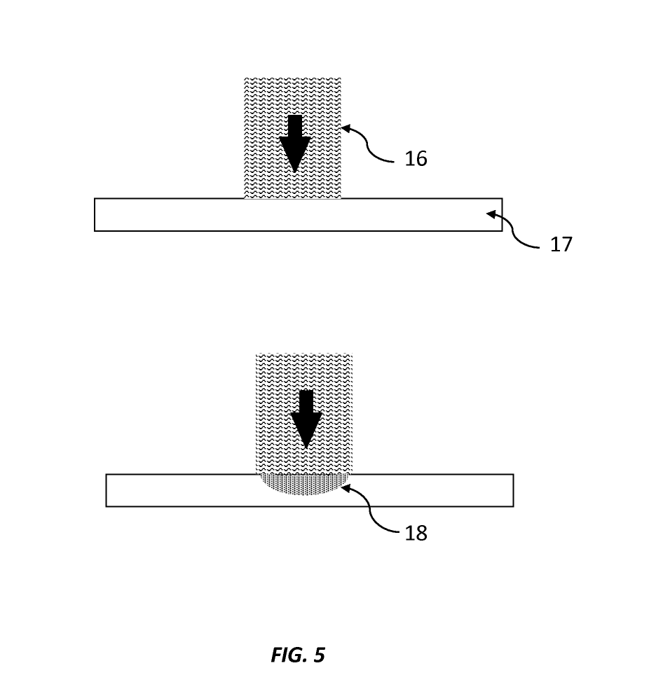

The first mechanism by which the non-equilibrium defect concentration in the near-surface layer on the metal medical implant can be created is through rapid thermal surface treatment. FIG. 5 shows the sequence of a heat source 16 interacting with the surface of the metal medical implant 17. A region of heated material 18 is created in the zone directly affected by the heat source.

The heat source can be one of the following: an ion beam, operating in pulsed or continuous mode; an electron beam, operating in pulsed or continuous mode; a laser, opeating in pulsed or continuous mode; or an electric arc, operating with straight or reverse polarity. The timeframe for the heat source to interact with the crystalline surface material must be fast. The on-heating equilibrium time for the vacancy concentration to reach the value for the elevated temperature is given by considering Equations 2 and 3 and can vary in general from as small as 10 nanoseconds at temperatures approaching the melting points of most common engineering metals, ceramics, and alloys to as large as 100 microseconds at temperatures at one half to one third of the melting point for most commonly used engineering metals, ceramics, and alloys. Therefore, the preferred characteristics of the heat source would be an interaction time of about 10 nanoseconds to about 100 microseconds. A short pulse is required to limit the depth of material that is heated or melted, so that the heat accumulated in the surface layer can be quickly dissipated. In continuous sources, this relevant time is the beam interaction time, i.e., the beam radius divided by the beam velocity. The time of pulsed heating or the interaction time with the beam must be small so as to limit the total volume of near-surface material that is heated and therefore to allow a quick quench. For this reason, isothermal heating of the entire implant followed by quench will not work, because the stored thermal energy in the bulk material of the implant below the surface layer becomes too large to allow a rapid cooling rate. Therefore, the goal is to minimize the stored thermal energy in the near-surface layer.

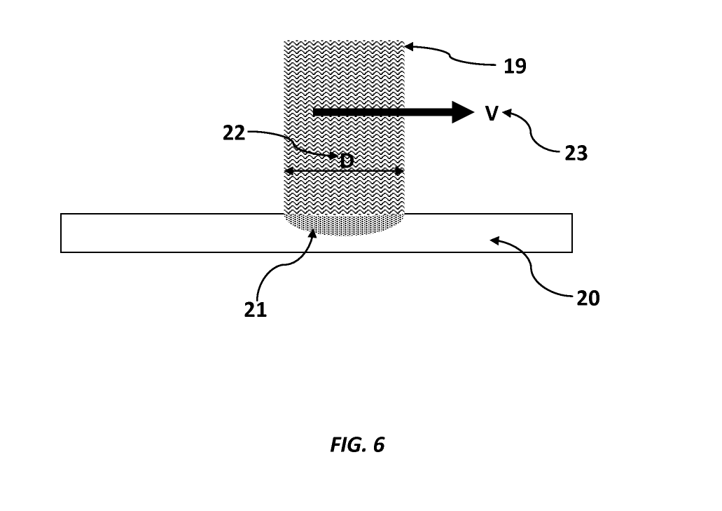

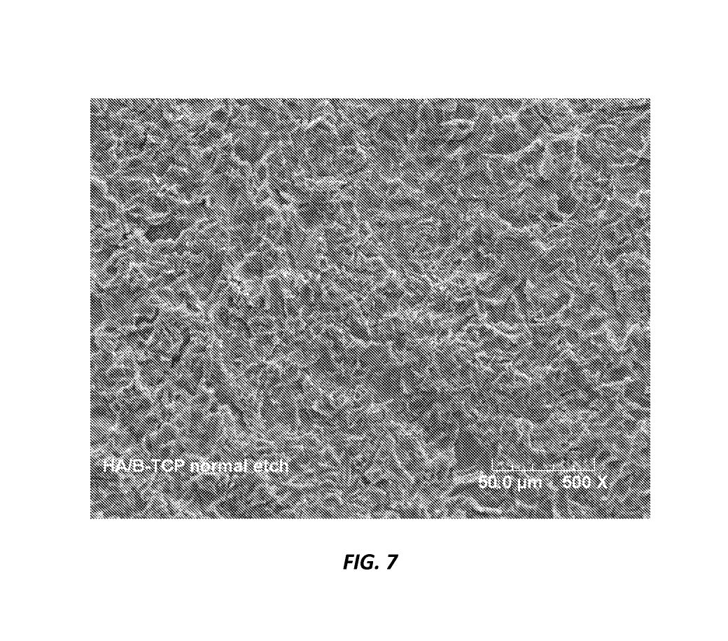

For continuous heat sources such as continuous electron beams, ion beams, lasers, or electric arcs, the situation is illustrated in FIG. 6. It is assumed that the continuous heat source 19 moves across the surface of the metal medical implant 20 and creates a transient region which is heated 21. The moving heat source has diameter 22 and travel speed 23 such that the velocity 23 will be fast enough to keep the interaction time small and the stored thermal energy in the near-surface region small as well. The interaction time is governed by the desired cooling rate, which must be about 10⁴ degrees K per second or greater. Therefore the relationship between travel velocity, beam diameter, cooling rate, and maximum temperature achieved may be estimated as follows. First, it is possible to approximate the cooling rate by relating it to the travel speed using a convective derivative:

$$\begin{matrix}\frac{\partial T}{\partial t} = cooling rate \\ = ( travel speed ) \cdot ( thermal gradient ) \\ = V \cdot \frac{\partial T}{\partial x}\end{matrix} \qquad ( 5 )$$

where V is the travel speed in the direction x, t is time, and T is temperature. It is possible to approximate the thermal gradient by considering the mathematical solution to the steady state thermal field produced by a travelling point heat source on a semi-infinite plane, which is given by Rosenthal (1946):

$$\begin{matrix}T - {T}_{0} = \frac{q}{2 π kR} \cdot exp [ \frac{- V ( R - x )}{2 α} ] \\ and & ( 6 ) \\ R = \sqrt{{x}^{2} + {y}^{2} + {z}^{2}} & ( 7 )\end{matrix}$$

where T is the temperature at location R from the center of the heat source, R is the distance which in this case will be set to the radius of the heat source (i.e., D/2), k is the thermal conductivity of the material, q is the power level of the heat source, V is the travel speed of the heat source, t is the time, and x is the distance along the axis the heat source is moving. Differentiating with respect to x and we get the thermal gradient as:

$$\frac{\partial T}{\partial x} = \frac{- q}{2 π kR} \cdot exp \{ \frac{- V}{2 α} \cdot ( R - x ) \} \cdot \{ \frac{x}{{R}^{2}} + ( \frac{V}{2 α} ) \cdot [ \frac{x}{R} - 1 ] \} \qquad ( 8 )$$

Evaluating this relationship at x=D/2, y=0, z=0:

$$\frac{\partial T}{\partial x} = \frac{- 2 q}{π k {D}^{2}} \qquad ( 9 )$$

So then, combining Equations 8 and 4 and taking the absolute value to get the magnitude, we get the relationship for the critical travel speed as a function of the desired cooling rate, the power of the heat source, and the diameter of the heat source:

$$V = {( \frac{\partial T}{\partial t} )}_{DESIRED} \cdot \frac{π {kD}^{2}}{2 q} \qquad ( 10 )$$

The desired cooling rate will be between 10⁴ and 10⁶ degrees Kelvin per second. The required travel speed can then be set based on Equation 9 for any given material or heat source type. Now we make one more substitution, which relates to the peak temperature that is to be achieved at the interface. Using Equation 5 and evaluating the rise in temperature at x=D/2, y=0, z=0, we get:

$$Δ T = \frac{q}{π kD} \qquad ( 11 )$$

Solving for q and substituting into Equation 9, we get:

$$V = {( \frac{\partial T}{\partial t} )}_{DESIRED} \cdot \frac{π {kD}^{2}}{2 \cdot Δ T \cdot π kD} = {( \frac{\partial T}{\partial t} )}_{DESIRED} \cdot \frac{D}{2 \cdot Δ T} \qquad ( 12 )$$

As an example, we can calculate the travel speed required to obtain a non-equilibrium concentration of vacancies using an electron beam to perform the rapid surface heat treatment. The following parameters would be used in the calculation: the desired cooling rate is 10⁵ degrees C./second; the diameter of the beam is 0.15 cm; and the temperature increase is 1200 degrees C. Then, the desired travel speed will be 8.3 cm/s. To get to a cooling rate of 10⁶ deg. C./sec, a travel speed of 83 cm/s would be needed. Therefore, according to Equation 11 it is possible to determine the required travel speed for any given beam power, temperature rise, beam diameter, and material type so that a non-equilibrium concentration of vacancies will be trapped in the near-surface layer of the metal. This actually applies for any metal in this approximate relationship since in equation 11 the “k” cancel out top and bottom. The “k” is implicit in the ΔT because the rise in temperature is related to the thermal properties of the material.

### EXAMPLE 1

As a specific example of how the present invention can produce the desired non-equilibrium structures on the surface of any metal implant, consider the following example in which

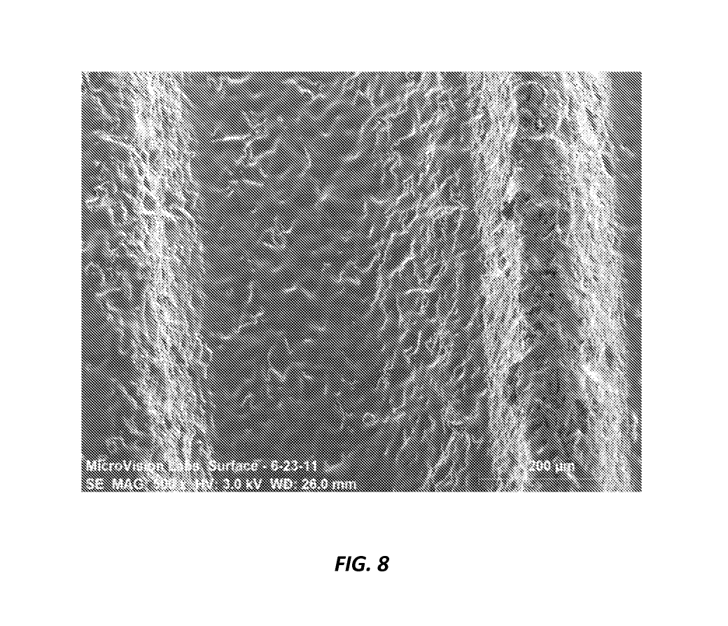

A commercially available titanium dental implant made out of commercially pure titanium Grade 4 was provided by Basic Dental Implant Inc. and was first subjected to media blast and acid etching in order to create a surface that is standard for current implants widely in use. The initial surface of the implant is shown in FIG. 7 and is shown after both the media blast and acid etch have taken place. The implant was then exposed to a pulsed ion beam with the following range of parameters:

| | |
|---|---|
| Beam Waveform (time) | 150 ns square pulse |
| Beam Waveform (spatial) | Gaussian |
| Beam Pulsewidth | 150 ns |
| Peak current | 20 kA |
| Peak Voltage | 350 kV |
| Average Energy Fluence at target in J/cm² | 4 J/cm² |

The resulting surface is shown in FIG. 8.

### EXAMPLE 2

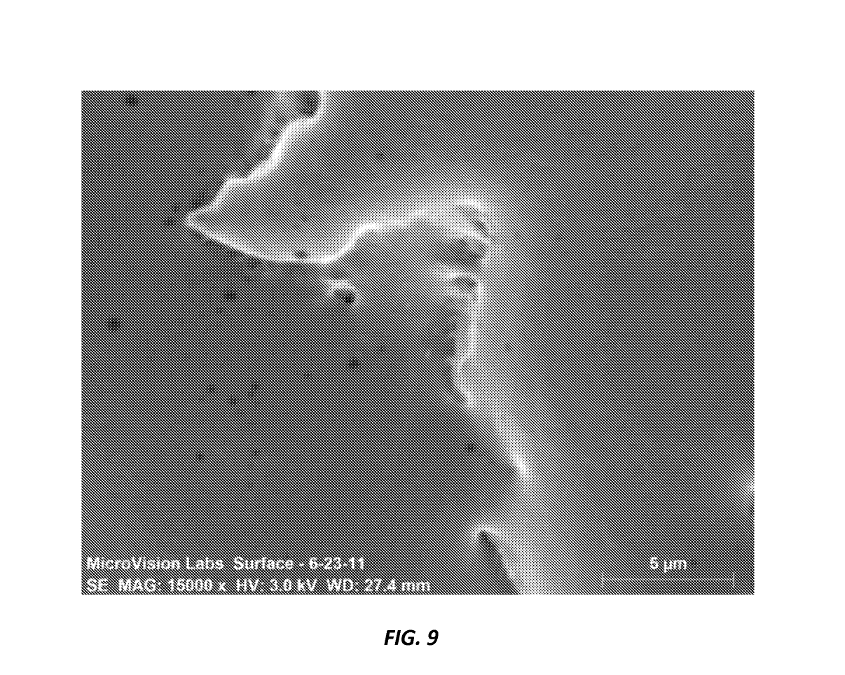

Implants made according to Example 1 were placed in human patients. Small batches of 6 implants were placed into 4 to 6 patients (some patients received more than 1 implant). The patients were recalled at 2 weeks and at four weeks following the surgery. Typical results were as follows. After 4 weeks, the bone did not show wound bone remodeling at all, and the implant (in all cases tested) showed nearly 100% osseointegration, which was demonstrated by the lack of a decrease in radio opacity at the implant surface during the healing period. There was also no invagination of any type at the crest of bone as typically seen in conventional titanium implants. The average osseointegration time was 30 days for one set of 6 patients, and there were greatly diminished clinical indications of pain and inflammation as observed by the implant medical care providers and as directly communicated by study subjects. When the surfaces of the implants placed in this study were examined under very high magnification (see FIG. 9), they were observed to be very smooth on a length scale of less than 10 micrometers, but had appreciable surface roughness retained from media blasting on length scales of 50-100 micrometers.

### REFERENCES

U.S. Pat. No. RE37,718 Blanchard et al
U.S. Pat. No. 8,007,850 Wei et al
U.S. Pat. No. 7,998,523 Lerf et al
U.S. Pat. No. 7,995,512 Park et al
U.S. Pat. No. 7,981,461 Georgette
U.S. Pat. No. 7,972,648 Berckmans III
U.S. Pat. No. 7,951,412 Justin et al
U.S. Pat. No. 7,951,285 Zipprish
U.S. Pat. No. 7,931,943 Schwartz et al
U.S. Pat. No. 7,931,683 Weber et al
U.S. Pat. No. 7,901,727 Hoffmann et al
U.S. Pat. No. 7,901,462 Yang et al
U.S. Pat. No. 7,897,727 Farach-Carson et al
U.S. Pat. No. 7,896,926 Hunter et al
U.S. Pat. No. 7,883,548 Chappa et al
U.S. Pat. No. 7,868,078 Jarrell
U.S. Pat. No. 7,857,987 Beaty
U.S. Pat. No. 7,824,462 Webster et al
U.S. Pat. No. 7,815,963 Schwartz et al
U.S. Pat. No. 7,803,234 Yeung et al
U.S. Pat. No. 7,785,648 Rohanizadeh et al
U.S. Pat. No. 7,775,796 Ihde
U.S. Pat. No. 7,771,774 Berckmans III et al
U.S. Pat. No. 7,763,270 Hellerbrand et al
U.S. Pat. No. 7,670,579 Chow et al
U.S. Pat. No. 7,666,522 Justin et al
U.S. Pat. No. 7,662,190 steinemann et all
U.S. Pat. No. 7,648,726 Liu et al
U.S. Pat. No. 7,556,500 Ihde
U.S. Pat. No. 7,550,091 Beaty
U.S. Pat. No. 7,547,399 Beaty
U.S. Pat. No. 7,419,679 Kuboki
U.S. Pat. No. 7,413,806 Ong et al
U.S. Pat. No. 7,368,065 Yang et al
U.S. Pat. No. 7,341,756 Liu et al
U.S. Pat. No. 7,229,545 Sewing et al
U.S. Pat. No. 7,169,317 Beaty
U.S. Pat. No. 7,122,810 De Maeztu Martinez
U.S. Pat. No. 7,087,085 steinemann et all
U.S. Pat. No. 7,048,870 Ellingsen et al
U.S. Pat. No. 7,001,672 Justin et al
U.S. Pat. No. 6,994,883 Layrolle et al
U.S. Pat. No. 6,974,532 Le Geros et al
U.S. Pat. No. 6,969,474 Beaty
U.S. Pat. No. 6,921,811 Zamora et al
U.S. Pat. No. 6,726,718 Carlyle et al
U.S. Pat. No. 6,702,855 steinemann et all
U.S. Pat. No. 6,652,765 Beaty
U.S. Pat. No. 6,627,321 Ellingsen et al
U.S. Pat. No. 6,582,470 Lee et al
U.S. Pat. No. 6,569,489 Li
U.S. Pat. No. 6,464,889 Lee et al
U.S. Pat. No. 6,419,491 Ricci et al
U.S. Pat. No. 6,419,491 Ricci et al
U.S. Pat. No. 6,399,215 Zhu et al
U.S. Pat. No. 6,339,913 Fong et al
U.S. Pat. No. 6,270,347 Webster et al
U.S. Pat. No. 6,262,017 Dee et al
U.S. Pat. No. 6,207,218 Layrolle et al
U.S. Pat. No. 6,183,255 Oshida
U.S. Pat. No. 6,146,686 Leitao
U.S. Pat. No. 6,132,214 Suhonen et al
U.S. Pat. No. 6,095,817 Wagner et al
U.S. Pat. No. 6,033,582 Lee et al
U.S. Pat. No. 6,005,164 Johansson et al
U.S. Pat. No. 5,932,299 Katoot
U.S. Pat. No. 5,876,453 Beaty
U.S. Pat. No. 5,855,612 Ohthuki et al
U.S. Pat. No. 5,843,289 Lee et al
U.S. Pat. No. 5,820,374 Simmons et al
U.S. Pat. No. 5,763,092 Dee et al
U.S. Pat. No. 5,738,521 Valentini et al
U.S. Pat. No. 5,738,521 Dugot et al
U.S. Pat. No. 5,730,598 Story et al
U.S. Pat. No. 5,723,038 Scharnweber et al
U.S. Pat. No. 5,716,412 De Carlo
U.S. Pat. No. 5,667,385 Hansson et al
U.S. Pat. No. 5,612,049 Li et al
U.S. Pat. No. 5,571,188 Ellingsen et al
U.S. Pat. No. 5,543,209 Duquet et al
U.S. Pat. No. 5,496,374 Blanchard et al
U.S. Pat. No. 5,372,660 Davidson et al
U.S. Pat. No. 5,344,494 Davidson et al
U.S. Pat. No. 5,242,706 Cotell et al
U.S. Pat. No. 5,211,833 shirkanzadeh et al
U.S. Pat. No. 5,068,002 Monroe et al
U.S. Pat. No. 5,039,546 Chung
U.S. Pat. No. 4,871,578 Adam et al
U.S. Pat. No. 4,834,756 Kenna et al
Adell, R. et al. a 15-year study of osseointegrated implants in the treatment of the endentulous jaw. Int. J. of Oral Surg., 10, pp. 387-416.
Anil, S., et al. (2011). Dental Implant Surface Enhancement and Osseointegration, Implant Dentistry—A Rapidly Evolving Practice, Ilser Turkyilmaz (Ed.), ISBN: 978-953-307-658-4, InTech, Available from: www.intechopen.com/articles/show/title/dental-implant-surface-enhancement-and-osseointegration
Ballo, A. M., et al. (2011). Dental Implant Surfaces—Physicochemical Properties, Biological Performance, and Trends, Implant Dentistry—A Rapidly Evolving Practice, Ilser Turkyilmaz (Ed.), ISBN: 978-953-307-658-4, InTech, Available from: www.intechopen.com/articles/show/title/dental-implant-surfaces-physicochemical-properties-biological-performance-and-trends
Branemark P I et al (2010). Bone response to laser-induced micro-and nano-sized titanium surface features. Submitted to Nanomedicine. [CAN YOU UPDATE THIS?]
Branemark, P I, et al (1969). Intra-osseous anchorage of dental prostheses. I Experimental studies. Scand. J. of Plast. Reconstr. Surg., 3, pp. 81-100.
Branemark P I, et al (1977). Osseointegrated implants in the treatment of the endentulous law. Experience from a 10-year period. Scand. J. of Plast. Reconstr. Surg. Suppl., 16, pp. 1-132.
Brånemark, P. I. (1983). Osseointegration and its experimental background. Journal of Prosthetic Dentistry, Vol. 50, No. 3, pp. 399-410, ISSN 0022-3913
Branemark et al (1997). Biomechanical characterization of osseointegration during healing: an experimental in-vivo study in the rat. Biomaterials, 18, pp. 969-978.
Buser D. et al (2004). Enhanced bone apposition to a chemically modified titanium SLA titanium surface. J. Dent. Res., 83, pp. 529-533.
Carlslaw, H S and Jaeger, J C 1959. Conduction of heat in solids—Second Edition. Oxford University Press, Oxford, UK.
Carr A B et al (1995). Reverse torque failure of screw shaped implants in baboons: baseline data for abutment torque application. Int. J. Oral Maxillofac. Implant. 10, pp. 167-174
Cook S D et al (1987). Interface mechanics and histology of titanium and HA-coated titanium for dental implant applications. Int. J. Oral Maxillofac Implants. 2, pp. 15-22
Celletti R. et al (2006). Bone contact around osseointegrated implants: a histologic study of acid-etched and machined surfaces. J. Long Term Effects Med. Implants, 16, pp. 131-143.
Conforto, E. et al (2004). Crystallographic properties and mechanical behavior of titanium hydride layers grown on titanium implants. Phil. Mag., 84, pp. 631-645.
Cooper L F, et al (2006). Fluoride modification effects on osteoblast behavior and bone formation at TiO2 grit blasted cp titanium endosseous implants. Biomaterials, 27, pp. 926-936.
Dave, V R (1995) Electron-beam assisted materials fabrication. MIT Ph.D. Thesis. Course III—Materials Science and Engineering Dept.
Dee, K. (2002). An introduction to tissue-biomaterial interactions. John Wiley and Sons, New York.
Feibelman P. J., Esch S., Michely T. (1996) Phys. Rev. Lett., 77, p. 2257.
Frojd, V. et al (2008). Increased bone contact to a calcium incorporated oxidized commercially pure titanium implant: in-vivo study in rabbits. Int. J. Oral Maxillofac. Surg. 37, pp. 561-566.
Hall J and Lausmaa J (2000). Properties of a new porous oxide surface on titanium implants. Applied Osseointegration Research, 1, pp. 5-8.
Haasen, P. (1986) Physical Metallurgy-2n^{d} Edition. Cambridge University Press, p. 41
He, J and Sinnott, S B (2005). Ab initio calculations of intrinsic defects in rutile TiO₂. J. Am. Ceram. Soc., 88 , pp. 737-741
Kittel, C. (2005) Introduction to Solid State Physics—8th ed. Wiley, New York.
Klokkevold P R et al (2001). Early endosseous integration enhanced by dual acid etching of titanium: a torque removal study in the rabbit. Clin Oral Implants Res., 12, pp. 350-357.
Koehler, J S and Lund, C. (1965). Non-equilibrium situations involving vacancies in gold—Lattice Defects in Quenched Metals—Cotterill et al. (Ed.). Academic Press, New York.
Lausmaa J. (2001). Mechanical, thermal, chemical, and electrochemical surface treatment of titanium. Titanium in medicine: materials science, surface science, engineering, biological responses and medical applications. Brunette D M Ed., Springer-Verlag, Berlin. P. 1019.
Leslie, W C (1973). Microstructural effects of high strain rate deformation, in Metallurgical Effects at High Strain Rates, Plenum Press, New York, N.Y., p. 571.
Novikov II et al (1981). Experimental investigation of vacancy effects in pure metals. J. Eng. Phys. Thermophys., 39, pp. 1316-1319.
Omar O. et al (2010). Integrin and chemokine receptor gene expression in implant-adherent cells during early osseointegration. J. Mater. Sci. Mater. Med. 21, pp. 969-980.
Ong et al 2002. Bone response to radio frequency sputtered calcium phosphate implants and titanium implants in vivo. J. Biomed. Mater Res. 59, pp. 184-190.
Palmquist, A. et al. (2010). Biomechanical, histological, and ultrastructural analyses of laser micro- and nano-structured titanium alloy (Ti-6Al-4V) in the rabbit. J. Biomed. Mater. Res. A, 92, pp. 1476-1486.
Persson B. N. J. (1992) Surf. Sci. Rep., 15, p. 1.
Ramazanoglu, M. and Oshida, Y. (2011). Osseointegration and Bioscience of Implant Surfaces—Current Concepts at Bone-Implant Interface, Implant Dentistry—A Rapidly Evolving Practice, Ilser Turkyilmaz (Ed.), ISBN: 978-953-307-658-4, InTech, Available from: www.intechopen.com/articles/show/title/osseointegration-and-bioscience-of-implant-surfaces-current-concepts-at-bone-implant-interface
Rechendorff, K. (2006). The influence of surface roughness on protein adsorption. Ph.D. Thesis, Univ. of Aarhus, Denmark.
Rosenberg et al (1991). Microbial differences in 2 clinically distinct types of failures of osseointegrated implants. Clin Oral Implants Res. 2 pp. 135-144
Rosenthal, D. 1946. The theory of moving heat source and its application to metal treatment. Trans ASME, 43, pp. 849-866.
Rupp, F. et al (2006). Enhancing surface free energy and hydrophillicity through chemical modification of microstructured titanium implant surfaces. J. Biomed. Mater. Res. A., 76, pp. 323-334.
Schwarz, F. et al. (2007). Bone regeration in dehiscence-type defects at chemically modified (SLActive) and conventional SLA titanium implants: a pilot study in dogs. J. Clin Periodontol. 34, pp. 78-86.
Seitz F and Koehler J S (1956). Solid State Phys. 2, pp. 305-448.
Silvennoinen, R., et al. (2011). Optical Detection of Protein Adsorption on Doped Titanium Surface, Biomaterials Science and Engineering, Rosario Pignatello (Ed.), ISBN: 978-953-307-609-6, InTech, Available from: www.intechopen.com/articles/show/title/optical-detection-of-protein-adsorption-on-doped-titanium-surface
Stanford, C. M. & Schneider, G. B. (2004) Functional behavior of bone around dental implants*. Gerodontology, 21, 71-77.
Vakarin, E V (2003). Unbinding of surface defects under the defect-selective adsorption. Condensed Mater Phys., 6, pp. 541.549
Verheyen, C C et al (1993). In vitro study on the integrity of a hydroxtapatite coating when challenged with staphylococci. J Biomedical Mater Res. 27, pp. 775-781.
Vidigal G M et al (2009). Osseointegration of titanium alloy and HA-coated implants in healthy and ovariectomized animals: a histomorphometric study. Clin Oral Implants Res., 20, pp. 1272-7.
Weng, D. et al (2003). Osseotite vs. machined surface in poor bone quality. A study in dogs. Clin Oral Implants Res., 14, pp. 703-708.
Wennerberg et al (1995). A histomorphometric and removal torque study of screw-shaped titanium implants with three different surface topographies. Clin Oral Implants Res., 6, pp. 24-30
Wennerberg (1996). On the surface roughness and implant incorporation. Goeteborg, Goeteborg University.

We claim:

1. A method of treating a medical implant comprising a crystalline surface material to promote protein adsorption to said surface material, the method comprising the steps of:
heating the surface material to form a surface layer comprising crystal lattice defects; and
thermally quenching the surface layer so as to preserve a non-equilibrium concentration of said defects.

2. The method of claim 1, wherein the heating step is performed such that the crystalline surface material is heated to at least one-half of its melting temperature for a period of time in the range from about 10 nanoseconds to about 100 microseconds.

3. The method of claim 1, wherein the heating step is performed using a pulsed or continuous energy source selected from the group consisting of an ion beam, an electron beam, a laser, and an electric arc.

4. The method of claim 3, wherein the energy source is a pulsed ion beam having a beam voltage in the range from about 100 kV to about 10 MV, a beam current in the range from about 1 A to about 1000 A, and a pulse width from about 10 nanoseconds to about 100 microseconds.

5. The method of claim 3, wherein the energy source is a pulsed electron beam having a beam voltage in the range from about 100 kV to about 10 MV, a beam current in the range from about 1 A to about 1000 A, and a pulse width from about 10 nanoseconds to about 100 microseconds.

6. The method of claim 3, wherein the energy source is an electron beam welding apparatus with a beam voltage in the range from about 50 kV to about 150 kV and a beam current in the range from about 1 mA to about 1 A.

7. The method of claim 3, wherein the energy source is a pulsed laser with a pulse width from about 10 picoseconds to about 1 millisecond, a pulse energy from about 1 picojoule per pulse to about one joule per pulse, wavelength from about 375 nm to about 1550 nm, and a pulse repetition rate from a single pulse to about 1 million pulses per second.

8. The method of claim 3, wherein the energy source is an arc welding apparatus operating either in straight or reverse polarity, with a voltage in the range from about 1V to about 20V, a current in the range from about 0.1 A to about 100 A, and wherein the heating step is carried out in the presence of one or more inert shielding gases.

9. The method of claim 1, wherein the quenching is performed at a rate of at least 104 degrees K per second.

10. (canceled)

11. (canceled)

12. (canceled)

13. (canceled)

14. The method of claim 1, wherein the surface layer has a thickness in the range from about 10 nm to about 25 μm.

15. (canceled)

16. The method of claim 1, wherein crystalline surface material below said surface layer has an equilibrium concentration of crystal lattice defects.

17. (canceled)

18. The method of claim 1, wherein the method of treating removes surface features and surface voids having a size of about 10 microns or less.

19. (canceled)

20. (canceled)

21. (canceled)

22. A method of treating a medical implant comprising a crystalline surface material to promote protein adsorption to said surface material, the method comprising the step of: performing shock deformation of the surface to form a surface layer comprising a non-equilibrium concentration of crystal lattice defects.

23. The method of claim 22, wherein shock deformation is performed by generating a shock wave in the surface layer using a pulsed energy source selected from the group consisting of a laser, an ion beam, and an electron beam.

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. A medical implant comprising a bulk material and a surface layer; wherein the surface layer is disposed at a surface of the implant, and the bulk material is disposed beneath the surface layer; wherein the bulk material and the surface layer have essentially the same chemical composition; wherein the surface layer has a non-equilibrium concentration of crystal lattice defects; and wherein the bulk material has an equilibrium concentration of crystal lattice defects.

48. The medical implant of claim 47, wherein the surface layer and bulk materials comprise a crystalline material selected from the group consisting of titanium, titanium oxide, zirconium, zirconium oxide, aluminum oxide, and hydroxyapatite.

49. The medical implant of claim 47, wherein the surface layer and bulk materials consist essentially of metallic material or ceramic material.

50. (canceled)

51. (canceled)

52. (canceled)

53. (canceled)

54. (canceled)

55. (canceled)

56. (canceled)

57. (canceled)

58. (canceled)

59. The medical implant of claim 47, wherein the surface layer has a thickness in the range from about 10 nm to about 25 μm.

60. (canceled)

61. (canceled)

62. The medical implant of claim 47, wherein surface features and surface voids of the surface layer have a size of 5 microns or less.

63. (canceled)

64. (canceled)
